SEQUENCE LISTING (4 sequences)

SEQ ID NO: 1 - DNA, 51 bp, synthetic construct
  source 1..51: mol_type = other DNA
tccttgatacctgcgaccatccagttccactcagatgtgtataagagacag

SEQ ID NO: 2 - DNA, 51 bp, synthetic construct
  source 1..51: mol_type = other DNA
ctgtctcttatacacatctgagtggaactggatggtcgcaggtatcaagga

SEQ ID NO: 3 - DNA, 50 bp, synthetic construct
  source 1..50: mol_type = other DNA
ctcagatcctacaacgacgctctaccgatgaagatgtgtataagagacag

SEQ ID NO: 4 - DNA, 50 bp, synthetic construct
  source 1..50: mol_type = other DNA
ctgtctcttatacacatcttcatcggtagagcgtcgttgtaggatctgag

(19) United States
(12) Patent Application Publication
(10) Pub. No.: US 2026/0085343 A1
(43) Pub. Date: Mar. 26, 2026

(54) METHOD FOR LIGATING NUCLEIC ACID FRAGMENTS, METHOD FOR CONSTRUCTING SEQUENCING LIBRARY, AND USE

(71) Applicant: GENEMIND BIOSCIENCES COMPANY LIMITED, Shenzhen (CN)

(72) Inventors: Guangli GAN, Shenzhen (CN); Qunting LIN, Shenzhen (CN); Yunyang CHENG, Shenzhen (CN); Meng ZHANG, Shenzhen (CN); Gailing LI, Shenzhen (CN); Qin YAN, Shenzhen (CN); Luyang ZHAO, Shenzhen (CN)

(73) Assignee: GENEMIND BIOSCIENCES COMPANY LIMITED, Shenzhen (CN)

(21) Appl. No.: 19/340,794

(22) Filed: Sep. 25, 2025

Related U.S. Application Data
(63) Continuation of application No. 17/266,542, now Pat. No. 12,428,667.

(30) Foreign Application Priority Data
Aug. 7, 2018 (CN) 201810890245.4

(51) Int. Cl. C12Q 1/6806; C12Q 1/6869

(52) U.S. Cl. CPC C12Q 1/6806; C12Q 1/6869

(57) ABSTRACT

Disclosed are a method for ligating nucleic acid fragments, a method for constructing a sequencing library, and the use thereof. The method for ligating nucleic acid fragments includes ligating a first nucleic acid fragment and a second nucleic acid fragment in a mixed enzyme reaction system including a DNA ligase and an RNA ligase, wherein the first nucleic acid fragment is double-stranded DNA. The ligation efficiency of the method for ligating nucleic acid fragments can reach 85% or more, and by using this ligation method to construct a sequencing library, the yield of the effective library is significantly improved.

### CROSS-REFERENCE TO RELATED APPLICATIONS

This application is a continuation of U.S. patent application Ser. No. 17/266,542, filed on Feb. 5, 2021, which is a U.S. national stage under 35 U.S.C. § 371 of International Application No. PCT/CN2019/099360, filed on Aug. 6, 2019, which claims priority of Chinese Patent Application No. 201810890245.4, filed on Aug. 7, 2018, the entire contents of each of which are hereby incorporated by reference.

### SEQUENCE LISTING

The instant application contains a Sequence Listing which is submitted electronically in XML format and is hereby incorporated by reference in its entirety. The XML copy, created on Sep. 12, 2025, is named “2025 Sep. 12-Sequence List-20969-D013US01” and is 4,855 bytes in size.

### TECHNICAL FIELD

The present disclosure relates to the technical field of biology, and in particular to a method for nucleic acid fragment ligation, a method for constructing a sequencing library, and use thereof.

### BACKGROUND

With the development of sequencing technologies, sequencing libraries are widely used. In genetic engineering, it is often necessary to ligate two or more nucleic acid fragments, for example, ligating an adapter, typically a short blunt-ended or sticky-ended synthetic oligonucleotide fragment, to a DNA molecule. The ligation efficiency between the adapter and nucleic acids directly affects the quality of the constructed sequencing library.

### SUMMARY

The present disclosure provides a method for nucleic acid fragment ligation, which is easy to operate and high in efficiency. Also provided is a method for constructing a sequencing library and use thereof in sequencing.

In one aspect, the present disclosure provides a method for nucleic acid fragment ligation, comprising: ligating a first nucleic acid fragment and a second nucleic acid fragment in a mixed enzyme reaction system comprising a DNA ligase and an RNA ligase, wherein the first nucleic acid fragment is a double-stranded DNA.

In one embodiment, the RNA ligase is a T4 RNA ligase; and/or the DNA ligase is a DNA ligase which can catalyze the ligation of sticky-ended or blunt-ended double-stranded DNA, for example, a T4 DNA ligase and/or a Blunt/TA ligase.

In one embodiment, the first nucleic acid fragment is a phosphorylated nucleic acid fragment.

In one embodiment, the phosphorylation is performed using a polynucleotide kinase.

In one embodiment, the polynucleotide kinase is a T4 polynucleotide kinase.

In one embodiment, the mixed enzyme reaction system further comprises a polynucleotide kinase.

In one embodiment, the polynucleotide kinase is a T4 polynucleotide kinase.

In one embodiment, the second nucleic acid fragment is a double-stranded nucleic acid fragment.

In one embodiment, a molar ratio of the first nucleic acid fragment to the second nucleic acid fragment is 1:8 to 1:20.

In one embodiment, the molar ratio of the first nucleic acid fragment to the second nucleic acid fragment is 1:10.

In one embodiment, a final concentration of the RNA ligase in a ligation reaction system is 0.15 U/μL to 1 U/μL; and/or the final concentration of the polynucleotide kinase in a phosphorylation system is 0.3 U/μL to 1 U/μL.

In another aspect, the present disclosure provides a method for constructing a sequencing library, comprising acquiring a ligation product by the method for ligation of any of the above embodiments to obtain a sequencing library, and the first nucleic acid fragment comes from a sample under test.

In a third aspect, the present disclosure provides a sequencing library given by the library construction method of any of the above embodiments.

In a fourth aspect, the present disclosure provides a method for sequencing, comprising sequencing the sequencing library obtained by the method of any of the above embodiments.

In a fifth aspect, the present disclosure provides a kit for implementing the method of any of the above embodiments.

RNA ligases are enzymes for RNA ligation. In the method for nucleic acid fragment ligation provided by the present disclosure, the reaction system simultaneously comprises a DNA ligase and an RNA ligase, and surprisingly, the DNA ligation efficiency is obviously improved. The experimental result shows that in a condition without RNA ligase where only DNA ligase is used for double-stranded DNA fragment ligation, the ligation efficiency is only 38.14-77.5%; however, when an RNA ligase commonly used for the ligation between RNAs is added, the DNA fragment ligation efficiency reaches more than 85%. When a sequencing library is constructed by this method for ligation, the yield of the effective library is obviously improved.

### BRIEF DESCRIPTION OF THE DRAWINGS

FIG. 1 is a diagram showing the results of identifying the size of ligation product by Labchip in Example 1;

FIG. 2 is a diagram showing the results of identifying the size of ligation product by Labchip in Example 2;

FIG. 3 is a diagram showing the results of identifying the size of ligation product by Labchip in Example 3;

FIG. 4 is a diagram showing the results of identifying the size of ligation product by Labchip in Example 4;

FIG. 5 is a diagram showing the results of identifying the size of ligation product by Labchip in Example 5;

FIG. 6 is a diagram showing the results of identifying the size of ligation product by Labchip in Example 6;

FIG. 7 is a diagram showing the results of identifying the size of ligation product by Labchip in Example 7;

FIG. 8 is a diagram showing the results of identifying the size of ligation product by Labchip in Example 8;

FIG. 9 is a diagram showing the results of identifying the size of ligation product by Labchip in Example 9;

FIG. 10 is a diagram showing the results of identifying the size of ligation product by Labchip in Example 10;

FIG. 11 is a diagram showing the results of identifying the size of ligation product by Labchip in Comparative Example 1; and

FIG. 12 is a diagram showing the results of identifying the size of ligation product by Labchip in Comparative Example 2.

### DETAILED DESCRIPTION

The aforementioned purposes, features, and advantages of the present disclosure will be more apparent from the description of the embodiments and drawings below. The following examples are illustrative only and are not to be construed as limiting the present disclosure.

In one aspect, the present disclosure provides a method for nucleic acid fragment ligation, comprising: ligating a first nucleic acid fragment and a second nucleic acid fragment in a mixed enzyme reaction system comprising a DNA ligase and an RNA ligase, wherein the first nucleic acid fragment is a double-stranded DNA.

Specifically, the first nucleic acid fragment may be a DNA fragment derived from cfDNA, genomic DNA, and/or PCR product, and the fragment may be blunt/end-filled or not.

Furthermore, the first nucleic acid fragment is a phosphorylated nucleic acid fragment.

Specifically, the first nucleic acid fragment is phosphorylated using a polynucleotide kinase.

Furthermore, the polynucleotide kinase is a T4 polynucleotide kinase.

In one embodiment, the mixed enzyme reaction system further comprises a polynucleotide kinase. The polynucleotide kinase phosphorylates the first nucleic acid fragments in a ligation process in a mixed enzyme system, thereby simplifying the operation and improving efficiency.

Specifically, the polynucleotide kinase in the mixed enzyme system is a T4 polynucleotide kinase. The T4 polynucleotide kinase (T4 PNK) can phosphorylate the ends of a double-stranded DNA fragment to form a fragment with a phosphate group at the 5′ ends.

Specifically, the second nucleic acid fragment is a double-stranded nucleic acid fragment having a known sequence, for example, an adapter. The adapter is a nucleotide fragment of a known sequence.

Furthermore, the adapter comprises a forward strand and a reverse strand, wherein the forward strand has an amino modification at the 5′ end and a hydroxyl modification at the 3′ end, and the reverse strand has a phosphorylation modification at the 5′ end, and a fluorescent label at the 3′ end. The 5′ end of the reverse strand can be ligated to the first nucleic acid fragment, and the forward and reverse strands form the double-stranded adapter.

Further, the adapter may be a blunt-ended adapter, a sticky-ended adapter, or the like.

Specifically, the fluorescent label may be a detectable fluorescent label such as CY3, TRITC, or TAMRA.

In one embodiment, the RNA ligase is a T4 RNA ligase. The T4 RNA ligase is commonly used for the ligation between RNAs. The inventor surprisingly found that when the T4 RNA ligase is used in the ligation between a double-stranded DNA and an adapter, it works well with T4 PNK and the T4 DNA ligase, achieving a ligation efficiency over 85% and a high yield of effective library.

Furthermore, the DNA ligase may be a T4 DNA ligase and/or a Blunt/TA ligase, etc., for ligating blunt-ended or sticky-ended DNAs.

In this embodiment, the DNA ligase is a T4 DNA ligase which catalyzes the formation of phosphodiester bonds between adjacent 3′ hydroxyl and 5′ phosphate ends on the double-stranded DNA and facilitates the ligation of the double-stranded DNA fragment to the adapter.

In one embodiment, the first nucleic acid fragment is a phosphorylated nucleic acid fragment.

In one embodiment, the phosphorylation is performed using a polynucleotide kinase.

In one embodiment, the molar ratio of the first nucleic acid fragment to the second nucleic acid fragment is 1:8 to 1:20. The molar ratio of the first nucleic acid fragment to the second nucleic acid fragment is appropriate for reducing the residue of the second nucleic acid fragment effectively.

Furthermore, the molar ratio of the first nucleic acid fragment to the second nucleic acid fragment is 1:10.

In one embodiment, the final concentration of the RNA ligase in the ligation reaction system is 0.15 U/μL to 1 U/μL; and/or

Furthermore, the final concentration of the polynucleotide kinase in the phosphorylation system is 0.3 U/μL to 1 U/μL.

In this embodiment, the ligation of the double-stranded DNA and the adapter specifically comprises the following steps: incubating the double-stranded DNA fragment in a mixed enzyme reaction system comprising a T4 polynucleotide kinase (T4 PNK), the T4 DNA ligase, and the T4 RNA ligase for ligation, and co-incubating the double-stranded DNA fragment and the adapter for ligation to give a ligation product.

Specifically, DNA may be fragmented by restriction enzymes, mechanical disruption, or the like, to form DNA fragments of appropriate length to facilitate ligation of the DNA fragments to the adapters. Certainly, if the length of the double-stranded DNA itself is within the appropriate length range or smaller than the appropriate length, the fragmentation is not required. The appropriate length may vary based on the sequencing platform. Before the randomly fragmented DNA fragments are ligated to the adapter, a filling-in treatment is usually performed, or the filling-in and ligation are performed simultaneously under the action of DNA ligase. However, for the present disclosure, the reaction system is suitable for the ligation of randomly fragmented DNA fragments without the filling-in process. Preferably, the T4 RNA ligase used is a T4 RNA ligase I.

In one embodiment, the ligation reaction is conducted in a two-step process as follows: incubating the double-stranded DNA fragment in a mixed enzyme system consisting of a T4 polynucleotide kinase and a T4 DNA ligase buffer for reaction for 20-40 min at 35-40° C. to give an intermediate product, the ends of the double-stranded DNA fragment being modified by phosphorylating with a T4 polynucleotide kinase (T4 PNK); and co-incubating the intermediate product and the adapter in a mixed enzyme system consisting of a T4 DNA ligase, a T4 RNA ligase, and a T4 DNA ligase buffer for 20-40 min at 10-20° C. to give a ligation product.

In another embodiment, the ligation reaction is conducted in a one-step process as follows: co-incubating the double-stranded DNA fragment and the adapter in a reaction system consisting of a T4 polynucleotide kinase, a T4 DNA ligase, a T4 RNA ligase, and a T4 DNA ligase buffer for 20-40 min at 10-20° C. to give a ligation product. The one-step process has similar ligation efficiency to the two-step process, but is easy to operate.

Specifically, the molar ratio of the double-stranded DNA fragment to the adapter is 1:8-1:20. The molar ratio of the double-stranded DNA fragment to the adapter is appropriate for reducing the adapter residue effectively.

Furthermore, the molar ratio of the double-stranded DNA fragment to the adapter is 1:10. As such, the amount of the adapter residue is minimized.

In the above method for ligation, an RNA ligase commonly used for ligating RNAs is added. The ligation efficiency of the double-stranded DNA fragments is improved by the cooperation of DNA ligase and RNA ligase. When the method for ligation is used for constructing a sequencing library, the effective library yield is elevated.

In another aspect, the present disclosure provides a method for constructing a sequencing library, comprising acquiring a ligation product by the method for ligation of any of the above embodiments to obtain a sequencing library, and the first nucleic acid fragment comes from a sample under test.

The method for constructing a sequencing library features the advantages of convenience, cost-efficiency, and high yield of an effective library.

In a third aspect, the present disclosure provides a sequencing library obtained by the method for construction of any of the above embodiments. In a fourth aspect, the present disclosure provides a method for sequencing, comprising performing a sequencing procedure on the sequencing library obtained by the method of any of the above embodiments.

In a fifth aspect, the present disclosure provides a kit for implementing the method of any of the above embodiments.

For example, the kit comprises a DNA ligase, an RNA ligase, and a package insert of the kit describing the method for ligation.

In one embodiment, the kit comprises a DNA ligase and an RNA ligase stored in the form of mixed enzymes.

In one embodiment, the kit further comprises an adapter.

The kit according to any of the above embodiments can be used to implement the efficient ligation of DNA fragments.

In the following examples, unless otherwise stated, the procedures without specifying the specific conditions are usually conducted according to conventional conditions, for example, see the Molecular Cloning: A Laboratory Manual [M] (Beijing Scientific Press, 1992) by Sambuque, E F Friedel, T Mannich, et. al. (translated by Jin Dongyan, Li Mengfeng, et. al.) or the method recommended by the kit manufacturer. The reagents or instruments not provided with manufacturer are conventional and commercially available products. As used herein, the term “and/or” includes any and all combinations of one or more of the associated listed items.

In the following examples:

The T4 Polynucleotide kinase (T4 PNK) was purchased from NEB, Cat. #M0201S, 10,000 U/mL.

The T4 DNA ligase was purchased from NEB, Cat. #M0202. The T4 RNA ligase I was purchased from NEB, Cat. #NEB M0204, 10,000 U/mL. Certainly, in other examples, ligases of other brands, such as Blunt/TA ligase mix of Vazyme Biotech Inc., may be used for ligation.

The synthetic sequences design is as follows:

(SEQ ID No. 1)
TCCTTGATACCTGCGACCATCCAGTTCCACTCAGATGTGTATAAGAGAC
AG;
(SEQ ID No. 2)
CTGTCTCTTATACACATCTGAGTGGAACTGGATGGTCGCAGGTATCAAG
GA;
(SEQ ID No. 3)
CTCAGATCCTACAACGACGCTCTACCGATGAAGATGTGTATAAGAGACA
G;
and
(SEQ ID No. 4)
CTGTCTCTTATACACATCTTCATCGGTAGAGCGTCGTTGTAGGATCTGAG

### D9 Adapter:

D9-S1 (forward strand): a sequence set forth in SEQ ID No. 1 with a modified amino group (—NH₂) at the 5′ end;
D9-S2 (reverse strand 1): a sequence set forth in SEQ ID No. 2 with a modified phosphate group (PHO-) at the 5′ end and an amino group (—NH₂) at the 3′ end;
D9-S2-CY3 (reverse strand 2): a sequence set forth in SEQ ID No. 2 with a modified phosphate group (PHO-) at the 5′ end and a fluorescent group (-CY3) at the 3′ end;
D9-1 adapter: a double-stranded polynucleotide formed by sequences D9-S1 (forward strand) and D9-S2-CY3 (reverse strand 2); and
D9-2 adapter: a double-stranded polynucleotide formed by sequences D9-S1 (forward strand) and D9-S2-CY3 (reverse strand 1).

### D7 Adapter:

D7-S1 (forward strand): a sequence set forth in SEQ ID No. 3 with a modified amino group (—NH₂) at the 5′ end;
D7-S2 (reverse strand 1): a sequence set forth in SEQ ID No. 4 with a modified phosphate group (PHO-) at the 5′ end and an amino group (—NH₂) at the 3′ end;
D7-S2 (reverse strand 2): a sequence set forth in SEQ ID No. 4 with a modified phosphate group (PHO-) at the 5′ end and a fluorescent group (-CY3) at the 3′ end;
D7-1 adapter: a double-stranded polynucleotide formed by sequences D7-S1 (forward strand) and D7-S2 (reverse strand 2); and
D7-2 adapter: a double-stranded polynucleotide formed by sequences D7-S1 (forward strand) and D7-S2 (reverse strand 1).

Adapter mix (adapter): a mixture of D7-1 adapter and D9-1 adapter in a molar ratio of 1:1.

### CY3: CY3 Fluorescent Dye

The molar ratio of DNA to adapter is the molar ratio of total DNA to adapter mix. In the examples, DNA refers to the PCR product.

D7-2 adapter and D9-2 adapter: the control for ligation product identification by Labchip.

The calculation of the adapter proportion:

Moles of DNA were calculated by the following formula (I):

$$pmols = \frac{m * {10}^{3}}{660 * L} ,$$

m: a weight of DNA fragment added into the system, in ng;
L: a length of DNA fragment added into the system, in bp.

Moles of adapter were calculated by the following formula (II):

$$pmols = C * V , \qquad ( II )$$

C: a molar concentration of adapter, in μM;
V: a volume of adapter used, in μL.

Moles given by the formula (I) were ratioed against those given by the formula (II) to afford the ratio of DNA to adapter. The ligation efficiency was calculated as follows: the adapter, the substrate, and the ligation product were separated by length using capillary electrophoresis on the Labchip platform; and picomoles of DNA with adapter were divided by the sum of picomoles of DNAs and multiplied by one hundred percent to give the ligation efficiency.

### Example 1

The method for ligation of the embodiment adopts a two-step process as follows:

15 ng of 200-bp PCR product, 2.5 μL of 10 μM ATP, 1 μL of 10× T4 ligase buffer (containing 10 mM ATP), and 1 μL of T4 PNK were mixed in a 200-μL PCR tube. The mixture was diluted to 10 μL using water, well mixed, and incubated at 37° C. for 30 min to give an intermediate product. 1 μL of 10× T4 ligase buffer, 2 μL of 1 μM adapter mix (adapter; the molar ratio of DNA to adapter was 1:17.6), 1 μL of T4 RNase ligase I, and 2 μL of T4 DNA ligase were added to the intermediate product. The mixture was diluted to 20 μL using water, well mixed (the final concentration of T4 RNase ligase I being 0.5 U/μL), and incubated for 30 min at 16° C. to give the ligation product.

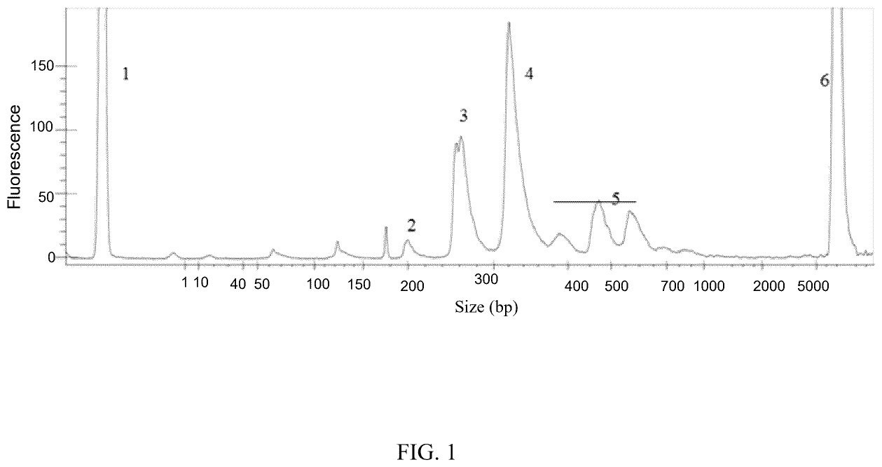

The resulting mixture was purified using 0.8× Ampure XP beads and was loaded on a Labchip platform to identify the size of ligation product. The results are shown in Table 1 and FIG. 1.

TABLE 1

| | | Molar concentration | |
|---|---|---|---|
| No. | Item | (pmol/mL) | % |
| 1 | Low marker | / | / |
| 2 | 200-bp PCR product | 0.12 | 4.14% |
| 3 | Product with one adapter | 0.95 | 32.76% |
| 4 | Product with two adapters | 1.38 | 47.59% |
| 5 | Polymers with adapter | 0.45 | 15.52% |
| 6 | Up marker | / | / |
| / | Effective library | 2.78 | 95.86% |
| / | Total | 2.9 | / |
| Adapter | / | / | 10.5% |
| residue | | | |

### Example 2

The method for ligation of the embodiment adopts a one-step process as follows:

15 ng of 200-bp PCR product, 2 μL of 10× T4 ligase buffer, 2 μL of 1 μM adapter mix (adapter; the molar ratio of DNA to adapter was 1:17.6), 1 μL of T4 PNK, 1 μL of T4 RNase ligase I, and 2 μL of T4 DNA ligase were mixed in a 200-μL PCR tube. The mixture was diluted to 20 μL using water, well mixed (the final concentration of T4 RNase ligase I being 0.5 U/μL), and incubated for 1 h at 16° C. to give the ligation product.

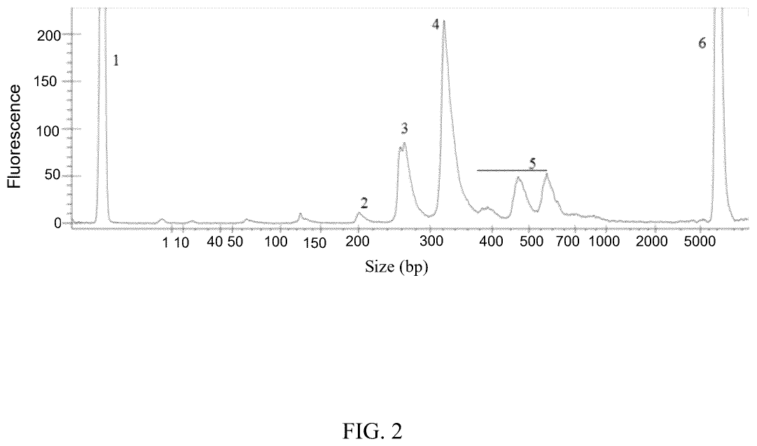

The resulting mixture was purified using 0.8× Ampure XP beads and was loaded on a Labchip platform to identify the size of ligation product. The results are shown in Table 2 and FIG. 2.

TABLE 2

| | | Molar concentration | |
|---|---|---|---|
| No. | Item | (pmol/mL) | % |
| 1 | Low marker | / | / |
| 2 | 200-bp PCR product | 0.11 | 3.68% |
| 3 | Product with one adapter | 0.84 | 28.09% |
| 4 | Product with two adapters | 1.53 | 51.17% |
| 5 | Polymers with adapter | 0.51 | 17.06% |
| 6 | Up marker | / | / |
| / | Effective library | 2.88 | 96.32% |
| / | Total | 2.99 | / |
| Adapter | / | / | 9.10% |
| residue | | | |

### Example 3

The method for ligation of the embodiment adopts a one-step process as follows:

15 ng of 200-bp PCR product, 2 μL of 10× T4 ligase buffer, 2 μL of 1 μM adapter mix (adapter; the molar ratio of DNA to adapter was 1:17.6), 1 μL of T4 PNK, 1 μL of T4 RNase ligase I, and 2 μL of T4 DNA ligase were mixed in a 200-μL PCR tube. The mixture was diluted to 20 μL using water, well mixed (the final concentration of T4 RNase ligase I being 0.5 U/μL), and incubated for 1 h at 16° C. to give the ligation product.

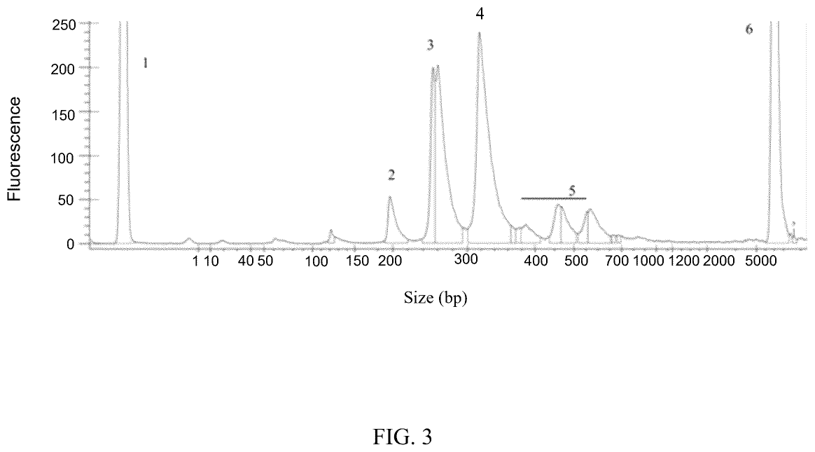

The resulting mixture was purified using 0.8× Ampure XP beads and was loaded on a Labchip platform to identify the size of ligation product. The results are shown in Table 3 and FIG. 3.

TABLE 3

| | | Molar concentration | |
|---|---|---|---|
| No. | Item | (pmol/mL) | % |
| 1 | Low marker | / | / |
| 2 | 200-bp PCR product | 0.39 | 9.63% |
| 3 | Product with one adapter | 1.74 | 42.96% |
| 4 | Product with two adapters | 1.44 | 35.56% |
| 5 | Polymers with adapter | 0.48 | 11.85% |
| 6 | Up marker | / | / |
| / | Effective library | 3.66 | 90.37% |
| / | Total | 4.05 | / |
| Adapter | / | / | 8.8% |
| residue | | | |

### Example 4

The method for ligation of the embodiment adopts a one-step process as follows:

The reaction system was prepared in the same manner as in Example 3, except that 1.7 μL of 1 μM adapter mix (adapter) was added and the molar ratio of DNA to adapter was 1:15. The rest was consistent with Example 3.

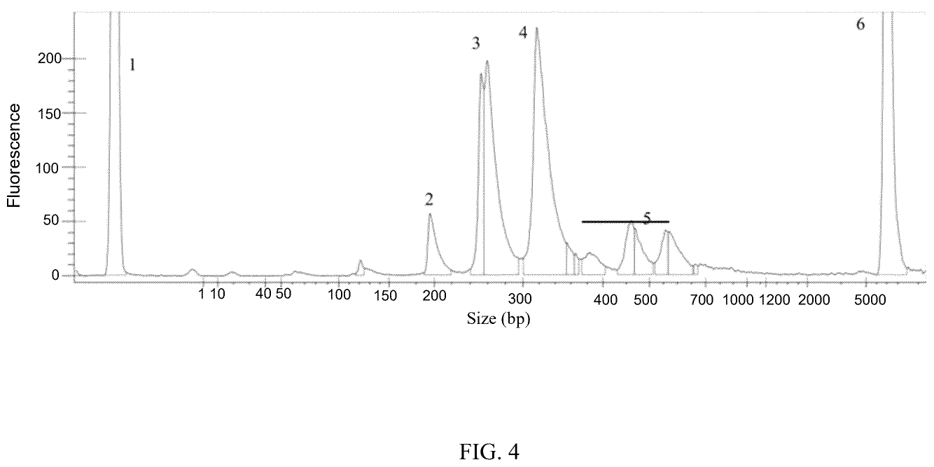

The resulting mixture was purified using 0.8× Ampure XP beads and was loaded on a Labchip platform to identify the size of ligation product. The results are shown in Table 4 and FIG. 4.

TABLE 4

| | | Molar concentration | |
|---|---|---|---|
| No. | Item | (pmol/mL) | % |
| 1 | Low marker | / | / |
| 2 | 200-bp PCR product | 0.41 | 10.43% |
| 3 | Product with one adapter | 1.65 | 41.98% |
| 4 | Product with two adapters | 1.37 | 34.86% |
| 5 | Polymers with adapter | 0.5 | 12.72% |
| 6 | Up marker | / | / |
| / | Effective library | 3.52 | 89.56% |
| / | Total | 3.93 | / |
| Adapter | / | / | 7.9% |
| residue | | | |

### Example 5

The method for ligation of the embodiment adopts a one-step process as follows:

The reaction system was prepared in the same manner as in Example 3, except that 1.36 μL of 1 μM adapter mix (adapter) was added and the molar ratio of DNA to adapter was 1:12. The rest was consistent with Example 3.

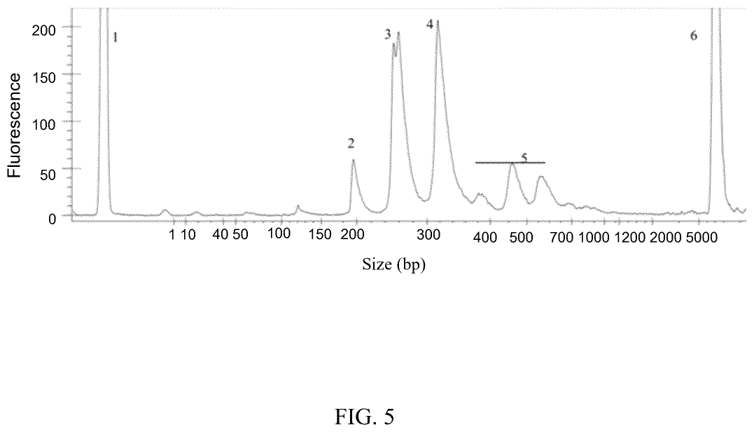

The resulting mixture was purified using 0.8× Ampure XP beads and was loaded on a Labchip platform to identify the size of ligation product. The results are shown in Table 5 and FIG. 5.

TABLE 5

| | | Molar concentration | |
|---|---|---|---|
| No. | Item | (pmol/mL) | % |
| 1 | Low marker | / | / |
| 2 | 200-bp PCR product | 0.44 | 11.17% |
| 3 | Product with one adapter | 1.68 | 42.64% |
| 4 | Product with two adapters | 1.26 | 31.98 |
| 5 | Polymers with adapter | 0.56 | 14.21% |
| 6 | Up marker | / | / |
| / | Effective library | 3.5 | 88.83% |
| / | Total | 3.94 | / |
| Adapter | / | / | 6.4% |
| residue | | | |

### Example 6

The method for ligation of the embodiment adopts a one-step process as follows:

The reaction system was prepared in the same manner as in Example 3, except that 1.14 μL of 1 μM adapter mix (adapter) was added and the molar ratio of DNA to adapter was 1:10. The rest was consistent with Example 3.

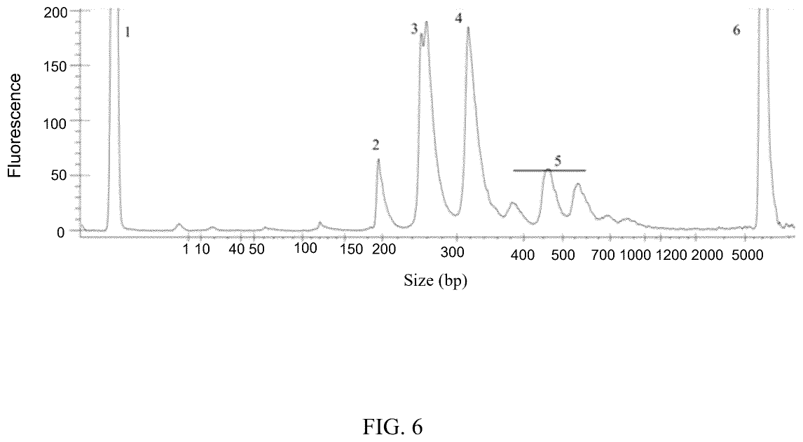

The resulting mixture was purified using 0.8× Ampure XP beads and was loaded on a Labchip platform to identify the size of ligation product. The results are shown in Table 6 and FIG. 6.

TABLE 6

| | | Molar concentration | |
|---|---|---|---|
| No. | Item | (pmol/mL) | % |
| 1 | Low marker | / | / |
| 2 | 200-bp PCR product | 0.46 | 12.2% |
| 3 | Product with one adapter | 1.62 | 42.97% |
| 4 | Product with two adapters | 1.11 | 29.44% |
| 5 | Polymers with adapter | 0.58 | 15.38% |
| 6 | Up marker | / | / |
| / | Effective library | 3.31 | 87.8% |
| / | Total | 3.77 | / |
| Adapter | / | / | 5.53% |
| residue | | | |

### Example 7

The method for ligation of the embodiment adopts a one-step process as follows:

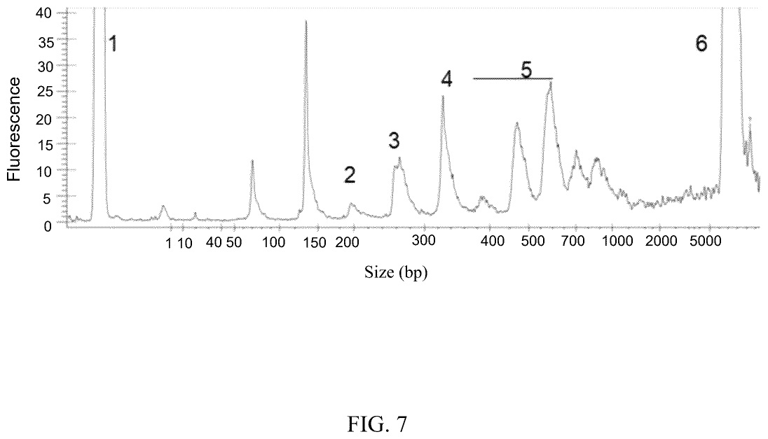

15 ng of 200-bp PCR product, 2 μL of 10× T4 ligase buffer, 1.14 μL of 1 μM adapter mix (adapter; the molar ratio of DNA to adapter was 1:10), 1 μL of T4 PNK, 1 μL of T4 RNase ligase I, and 2 μL of T4 DNA ligase were mixed in a 200-μL PCR tube. The mixture was diluted to 20 μL using water, well mixed (the final concentration of T4 RNase ligase I being 0.5 U/μL), and incubated for 30 min at 16° C. to give the ligation product. The resulting mixture was purified using 0.8× Ampure XP beads and was loaded on a Labchip platform to identify the size of ligation product. The results are shown in Table 7 and FIG. 7.

TABLE 7

| | | Molar concentration | |
|---|---|---|---|
| No. | Item | (pmol/mL) | % |
| 1 | Low marker | / | / |
| 2 | 200-bp PCR product | 0.03 | 5.77% |
| 3 | Product with one adapter | 0.11 | 21.15% |
| 4 | Product with two | 0.11 | 21.15% |
| | adapters | | |
| 5 | Polymers with adapter | 0.27 | 51.92% |
| 6 | Up marker | / | / |
| / | Effective library | 0.49 | 94.23% |
| / | Total | 0.52 | / |

### Example 8

The method for ligation of the embodiment adopts a one-step process as follows:

The reaction system was prepared in the same manner as in Example 7, except that 1 μL of T4 RNase ligase I and 1 μL of T4 DNA ligase were added. The rest was consistent with Example 7.

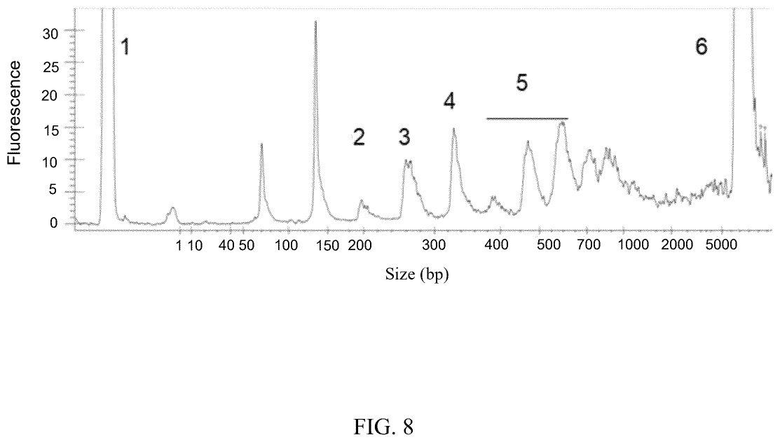

The resulting mixture was purified using 0.8× Ampure XP beads and was loaded on a Labchip platform to identify the size of ligation product. The results are shown in Table 8 and FIG. 8.

TABLE 8

| | | Molar concentration | |
|---|---|---|---|
| No. | Item | (pmol/mL) | % |
| 1 | Low marker | / | / |
| 2 | 200-bp PCR product | 0.01 | 3.22% |
| 3 | Product with one adapter | 0.07 | 22.58% |
| 4 | Product with two adapters | 0.06 | 19.35% |
| 5 | Polymers with adapter | 0.17 | 54.84% |
| 6 | Up marker | / | / |
| / | Effective library | 0.30 | 96.77% |
| / | Total | 0.31 | / |

### Example 9

The method for ligation of the embodiment adopts a one-step process as follows:

The reaction system was prepared in the same manner as in Example 7, except that 0.5 μL of T4 RNase ligase I and 0.5 μL of T4 DNA ligase were added, and the final concentration of T4 RNase ligase I was 0.25 U/μL. The rest was consistent with Example 7.

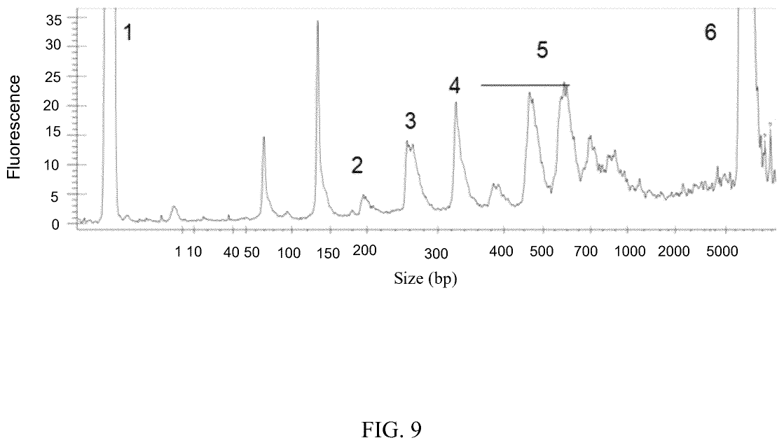

The resulting mixture was purified using 0.8× Ampure XP beads and was loaded on a Labchip platform to identify the size of ligation product. The results are shown in Table 9 and FIG. 9.

TABLE 9

| | | Molar concentration | |
|---|---|---|---|
| No. | Item | (pmol/mL) | % |
| 1 | Low marker | / | / |
| 2 | 200-bp PCR product | 0.02 | 4.35% |
| 3 | Product with one adapter | 0.11 | 23.91% |
| 4 | Product with two adapters | 0.08 | 17.39% |
| 5 | Polymers with adapter | 0.25 | 54.35% |
| 6 | Up marker | / | / |
| / | Effective library | 0.44 | 95.65% |
| / | Total | 0.46 | / |

### Example 10

The method for ligation of the embodiment adopts a one-step process as follows:

15 ng of 200-bp PCR product, 3 μL of 10× T4 ligase buffer, 1.14 μL of 1 μM adapter mix (adapter; the molar ratio of DNA to adapter was 1:10), 1 μL of T4 PNK, 0.5 μL of T4 RNase ligase I and 0.5 μL of T4 DNA ligase were mixed in a 200-μL PCR tube. The mixture was diluted to 30 μL using water, well mixed (the final concentration of T4 RNase ligase I being 0.17 U/L), and incubated for 15 min at 16° C. to give the ligation product.

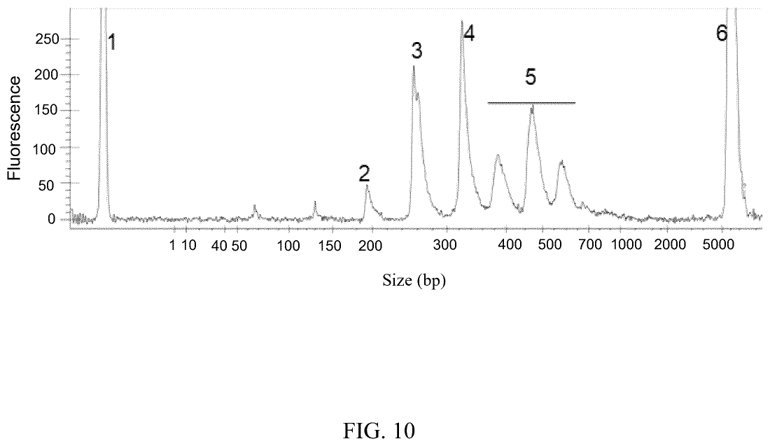

The resulting mixture was purified using 0.8× Ampure XP beads and was loaded on a Labchip platform to identify the size of ligation product. The results are shown in Table 10 and FIG. 10.

TABLE 10

| | | Molar concentration | |
|---|---|---|---|
| No. | Item | (pmol/mL) | % |
| 1 | Low marker | / | / |
| 2 | 200-bp PCR product | 0.33 | 7.24% |
| 3 | Product with one adapter | 1.51 | 33.11% |
| 4 | Product with two adapters | 1.25 | 27.41% |
| 5 | Polymers with adapter | 1.47 | 32.24% |
| 6 | Up marker | / | / |
| / | Effective library | 4.23 | 92.76% |
| / | Total | 4.56 | / |

### Comparative Example 1

The method for ligation of the embodiment adopts a two-step process as follows:

15 ng of 200-bp PCR product, 2.5 μL of 10 μM ATP, 1 μL of 10× T4 ligase buffer, and 1 μL of T4 PNK were mixed in a 200-μL PCR tube. The mixture was diluted to 10 μL using water, well mixed, and incubated at 37° C. for 30 min to give an intermediate product. 1 μL of 10× T4 ligase buffer, 2 μL of 1 μM adapter mix (adapter; the molar ratio of DNA to adapter was 1:17.6), and 2 μL of T4 DNA ligase were added to the intermediate product. The mixture was diluted to 20 μL using water, well mixed, and incubated for 30 min at 16° C. to give the ligation product.

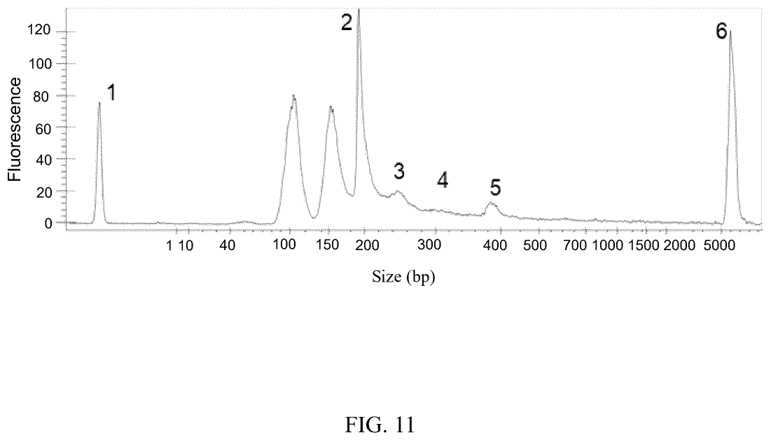

The resulting mixture was purified using 0.8× Ampure XP beads and was loaded on a Labchip platform to identify the size of ligation product. The results are shown in Table 11 and FIG. 11.

TABLE 11

| | | Molar concentration | |
|---|---|---|---|
| No. | Item | (pmol/mL) | % |
| 1 | Low marker | / | / |
| 2 | 200-bp PCR product | 6.44 | 61.86% |
| 3 | Product with one adapter | 2.38 | 22.86% |
| 4 | Product with two adapters | 0.98 | 9.41% |
| 5 | Polymers with adapter | 0.61 | 5.86% |
| 6 | Up marker | / | |
| / | Effective library | 3.97 | 38.14% |
| / | Total | 10.41 | |

### Comparative Example 2

The method for ligation of the embodiment adopts a one-step process as follows:

15 ng of 200-bp PCR product, 2 μL of 10× T4 ligase buffer, 2 μL of 1 μM adapter mix (adapter; the molar ratio of DNA to adapter was 1:17.6), 1 μL of T4 PNK, and 2 μL of T4 DNA ligase were mixed in a 200-μL PCR tube. The mixture was diluted to 20 μL using water, well mixed, and incubated for 1 h at 16° C. to give the ligation product.

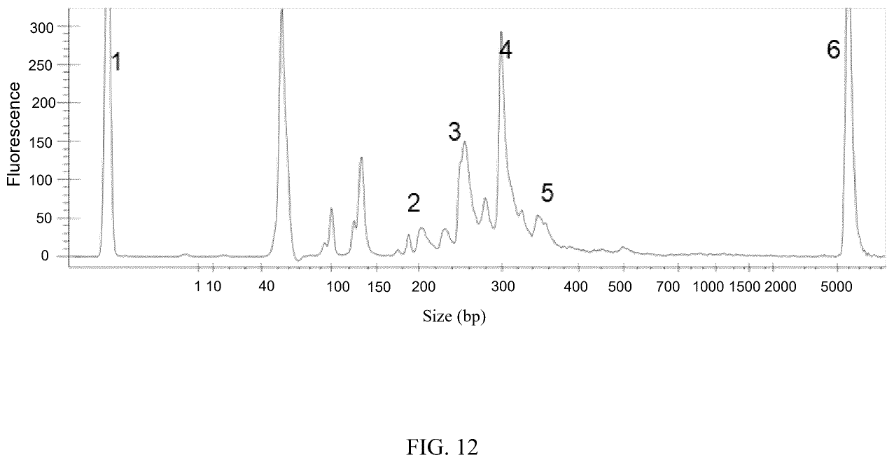

The resulting mixture was purified using 0.8× Ampure XP beads and was loaded on a Labchip platform to identify the size of ligation product. The results are shown in Table 12 and FIG. 12.

TABLE 12

| | | Molar concentration | |
|---|---|---|---|
| No. | Item | (pmol/mL) | % |
| 1 | Low marker | / | / |
| 2 | 200-bp PCR product | 0.76 | 11.71% |
| 3 | Product with one adapter | 2.25 | 40.83% |
| 4 | Product with two adapters | 2.07 | 34.98% |
| 5 | Polymers with adapter | 0.71 | 12.48% |
| 6 | Up marker | / | |
| / | Effective library | 5.03 | 77.5% |
| / | Total | 6.49 | |

The results of Comparative Examples 1 and 2 show that in a ligation reaction system without T4 RNA ligase I, the ligation efficiency is significantly reduced: in the two-step ligation process, the ligation efficiency was 38.14%, while in the one-step ligation reaction, the ligation efficiency was 77.5%.

The above results indicate that the method of Examples 1 to 10, in which T4 RNA Ligase I (T4 RNA ligase I) commonly used for the ligation between RNAs was added to the ligation of double-stranded DNA and the adapter, demonstrated an 85% or higher ligation efficiency of the double-stranded DNA fragment and the adapter and a high yield of effective library. The method for ligation requires no PCR process, and is convenient and cost-efficient.

Examples 2 to 10 adopt a one-step ligation process, which is easy to operate and is similar to the two-step process of Example 1 in efficiency.

Example 6, in which the molar ratio of DNA to adapter was 1:10, shows less adapter residue as compared with Examples 1 to 5.

Examples 7 to 9 show that at reduced doses of T4 RNA ligase I and T4 DNA ligase, the ligation efficiency is equivalent to that of the original doses. With a reduced amount of enzymes used in the experiment, the cost can be reduced.

In Example 10, when the system was scaled up to adapt the sample concentration, the reaction efficiency of the method for ligation of the present disclosure showed no significant reduction.

The above examples only illustrate one or more embodiments of the present disclosure for the purpose of specific and detailed description, but should not be construed as limiting the scope of the present disclosure. It should be noted that various changes and modifications can be made by those skilled in the art without departing from the spirit of the present disclosure, and these changes and modifications are all within the scope of the present disclosure. Therefore, the protection scope of the present disclosure should be determined with reference to the appended claims.

What is claimed is:

1. A method for nucleic acid fragment ligation, comprising:
ligating a first nucleic acid fragment and a second nucleic acid fragment in a mixed enzyme reaction system comprising a DNA ligase and an RNA ligase, wherein the first nucleic acid fragment is a double-stranded DNA.

2. The method as claimed in claim 1, wherein the mixed enzyme reaction system further comprises a polynucleotide kinase, wherein the DNA ligase is selected as a T4 DNA ligase, the RNA ligase is selected as a T4 RNA ligase, and the polynucleotide kinase is selected as a T4 polynucleotide kinase.

3. The method as claimed in claim 1, wherein the first nucleic acid fragment is a phosphorylated nucleic acid fragment.

4. The method as claimed in claim 2, wherein the polynucleotide kinase is used to perform a phosphorylation treatment.

5. The method as claimed in claim 1, wherein the second nucleic acid fragment is a double-stranded nucleic acid fragment.

6. The method as claimed in claim 5, wherein the second nucleic acid fragment comprises sequences of SEQ ID No. 1 and 2, and SEQ ID No. 3 and 4.

7. The method as claimed in claim 6, wherein SEQ ID No. 1 has a modified amino group at the 5′ end, SEQ ID No. 2 has a modified phosphate group at the 5′ end, SEQ ID No. 3 has a modified amino group at the 5′ end, and SEQ ID No. 4 has a modified phosphate group at the 5′ end.

8. The method as claimed in claim 6, wherein SEQ ID No. 1 and 2, and SEQ ID No. 3 and 4 are in a molar ratio of 1:1.

9. The method as claimed in claim 1, wherein a molar ratio of the first nucleic acid fragment to the second nucleic acid fragment is 1:8 to 1:20.

10. The method as claimed in claim 9, wherein the molar ratio of the first nucleic acid fragment to the second nucleic acid fragment is 1:10.

11. The method as claimed in claim 2, wherein a final concentration of the RNA ligase in a ligation reaction system is 0.15 U/μL to 1 U/μL.

12. The method as claimed in claim 11, wherein a final concentration of the polynucleotide kinase in a phosphorylation system is 0.3 U/μL to 1 U/μL.

13. The method as claimed in claim 2, further comprising:
mixing the first nucleic acid fragment, the second nucleic acid fragment, the T4 polynucleotide kinase, the T4 DNA ligase, and the T4 RNA ligase for 20-40 min at 10-20° C. to obtain a ligation product.

14. The method as claimed in claim 1, further comprising: subjecting DNA fragment in a mixed enzyme system consisting of a T4 polynucleotide kinase and a T4 DNA ligase buffer to a reaction for 20-40 min at 35-40° C. to give an intermediate product, and co-incubating the intermediate product and an adapter in a mixed enzyme system consisting of a T4 DNA ligase, a T4 RNA ligase, and a T4 DNA ligase buffer for 20-40 min at 10-20° C. to give a ligation product.

15. The method as claimed in claim 9, wherein an amount of the first nucleic acid fragment is calculated using formula (I):
$$pmols = \frac{m * {10}^{3}}{660 * L} , \qquad ( I )$$
wherein
m is a weight of the first nucleic acid fragment, in ng,
L is a length of the first nucleic acid fragment, in bp; and
an amount of the second nucleic acid fragment is calculated using formula (II):
$$pmols = C * V , \qquad ( II )$$
wherein
C is a molar concentration of the second nucleic acid fragment, in μM,
V is a volume of the second nucleic acid fragment, in μL.

16. A method for constructing a sequencing library, comprising:
acquiring a ligation product by the method as claimed in claim 1, to obtain the sequencing library, wherein the first nucleic acid fragment is from a sample under test.

17. A sequencing method, comprising:
acquiring a sequencing library using the method as claimed in claim 16, and sequencing the sequencing library.

18. A kit configured for performing the method as claimed in claim 1.

19. The kit as claimed in claim 18, comprising a DNA ligase, an RNA ligase, and a package insert of the kit describing the method for ligation.
